(19) United States
(12) Patent Application Publication
(10) Pub. No.: US 2015/0013684 A1
(43) Pub. Date: Jan. 15, 2015

(54) SPEAKING VALVE SYSTEM WITH CUFF DEFLATION

(71) Applicant: Covidien LP, Boulder, CO (US)

(72) Inventors: Kamlesh Subhash Sethiya, Longmont, CO (US); Emmet Gerard Bolger, Lyons, CO (US); John P. Burns, Thornton, CO (US); Brian Rosekrans, Co. Westmeath (IE); Sean M. Stephens, Co. Westmeath (IE)

(21) Appl. No.: 13/939,750

(22) Filed: Jul. 11, 2013

(51) Int. Cl. A61M 16/04; A61M 16/00; A61M 16/20

(52) U.S. Cl. CPC A61M 16/0468; A61M 16/044; A61M 16/202; A61M 16/0051; A61M 16/208; A61M 16/0003; A61M 2016/0027
USPC 128/207.15

(57) ABSTRACT

A tracheal tube system includes a tracheal tube assembly having a cannula configured to be positioned in a patient airway, and a connector coupled to the proximal end of the cannula. The tracheal tube assembly further includes a cuff disposed about the cannula. The tracheal tube system additionally includes a speaking valve comprising means for deflating the cuff, wherein the speaking valve, the connector, and the cannula form a contiguous passageway for delivering air one-way into the patient airway when the speaking valve is disposed onto the end connector.

### BACKGROUND

The present disclosure relates generally to the field of medical devices, and more particularly, to airway devices, such as speaking valve systems that include cuff deflation mechanisms.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of situations exist in which artificial ventilation of a patient may be desired. For short-term ventilation or during certain surgical procedures, endotracheal tubes may be inserted through the mouth to provide oxygen and other gasses to a patient. For certain applications, particularly when longer-term intubation is anticipated, tracheostomy tubes may be preferred. Tracheostomy tubes are typically inserted through an incision made in the neck of the patient and into the trachea. A resulting stoma is formed between the tracheal rings below the vocal chords. The tracheostomy tube is then inserted through the opening.

Such tubes may include an inner cannula and an outer cannula, with the inner cannula, may be disposed inside the outer cannula and used as a conduit for liquids or gas or medicine incoming and outgoing into the patient's lungs. The inner cannula may be removed for cleaning and for disposal of secretions while leaving the outer cannula in place, thus maintaining a desired placement of the tracheostomy tube. An inflatable cuff may be additionally provided, useful in securing the outer cannula to the patient airway and blocking fluid flow around the outer cannula, thus enabling the tracheal tube to serve as a sole artificial conduit into the airway. A connector is typically provided at an upper or proximal end where the tube exits the patient airway, suitable for coupling the ventilator with the inner cannula. A set of flanges or wings are disposed around the outer cannula and used to securely couple the tracheostomy tube to the patient neck.

To provide the patient the ability to breathe and speak, a one-way valve may be disposed over an end of the tracheostomy tube or connector that is external to the patient. Once in place, the one-way valve generally permits airflow to travel in only one direction within the tracheostomy tube. When the patient inhales, the check valve opens to allow air into the lungs. However, when the patient exhales, the check valve closes to enable the exhalation air to exit via the mouth and/or nose to facilitate speaking and breathing. When the one-way valve is in use, it may be desired to maintain the cuff in a deflated condition, thus enabling flow of air around the outer cannula and outwardly towards the vocal chords. There is a need, therefore, for improved speaking valves, particularly with respect to speaking valves that may deflate a cuff when in use.

### BRIEF DESCRIPTION

This disclosure provides a novel speaking valve system designed to respond to such needs. The speaking valve system may include mechanical techniques, electronic techniques, or a combination thereof, useful in deflating a cuff when the speaking valve is in use. By deflating the cuff during use of the speaking valve, air may exit from the lungs and traverse around the tracheal tube outwardly towards the vocal chords, improving speech and enhancing patient safety. In one example, a patient may be intubated and a cuff may be inflated, and, when the patient desires to speak, the speaking valve may be positioned onto a proximal end of the tracheal tube for use as a one-way check valve. The speaking valve system may include one or more protrusions suitable for mating with a receptacle of a lumen that includes an inflation valve used to inflate the cuff. By inserting a protrusion into the receptacle and using the protrusion to depress, for example, a head of the inflation valve, the cuff may be deflated.

In another example, the speaking valve may include a component such as a magnetic device, a radio frequency tag, a Bluetooth transmitter, a wireless transmitter, and the like, which may communicatively couple with an electronic cuff deflation device included in a tracheal tube system, such as a tracheostomy tube. The electronic cuff deflation device may detect the attachment of the speaking valve to the tracheostomy tube and may then deflate cuff(s) inflated about the tracheostomy tube. The speaking valve may further include visual indications that the cuff is or is not deflated. For example, light emitting diodes (LEDs) may be incorporated in the speaking valve and used to indicate the inflation status of the cuff(s). By providing for mechanical and/or electronic techniques for deflating the cuff(s) during use of the speaking valve, the techniques described herein may improve patient comfort, safety, and enhance phonation.

Thus, in accordance with a first aspect, a tracheal tube system includes a tracheal tube assembly having a cannula configured to be positioned in a patient airway, and a connector coupled to the proximal end of the cannula. The tracheal tube assembly further includes a cuff disposed about the cannula. The tracheal tube system additionally includes a speaking valve comprising means for deflating the cuff, wherein the speaking valve, the connector, and the cannula form a contiguous passageway for delivering air one-way into the patient airway when the speaking valve is disposed onto the end connector.

In accordance with another aspect, a tracheal tube system is provided. The tracheal tube system includes a speaking valve. The speaking valve includes a first protrusion comprising a first width and a first length, wherein the first width is smaller than a bore width of a bore included in a cuff deflation device, and the first length is longer than a distance between a proximal opening in the bore an a deflation head disposed in the bore, and wherein the speaking valve provides for one-way airway gas flow when in use.

Also disclosed herein is a tracheal tube system having a speaking valve. The speaking valve includes a communications module comprising a wireless circuitry configured to transmit a wireless cuff deflation signal to deflate a cuff, wherein the speaking valve provides for one-way airway gas flow when disposed onto a tracheal tube assembly having the cuff

### BRIEF DESCRIPTION OF THE DRAWINGS

Various aspects of the disclosed techniques may become apparent upon reading the following detailed description and upon reference to the drawings in which:

FIG. 1 is a perspective view of an exemplary tracheal tube assembly including a speaking valve having cuff deflation means, in accordance with aspects of present techniques;

FIG. 2 illustrates a patient having a tracheostomy system with the speaking valve of FIG. 1, according to embodiments of the present techniques;

FIG. 3 is a side view of the exemplary speaking valve of FIG. 1 showing the application of a cuff deflator;

FIG. 4 is a side view of the speaking valve of FIG. 1 showing a valve cap and a valve body having portions of the cuff deflator;

FIG. 5 a frontal view of the speaking valve of FIG. 1 showing the valve cap and the valve body having portions of the cuff deflator;

FIG. 6 is a frontal view of portions of the cuff deflator forming a circular shape;

FIG. 7 is a frontal view of portions of the cuff deflator forming a square shape;

FIG. 8 is a frontal view of portions of the cuff deflator forming a hexagonal shape; and

FIG. 9 is a block diagram of an electronic architecture of an exemplary electronic valve communications module and an electronic cuff deflation system.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

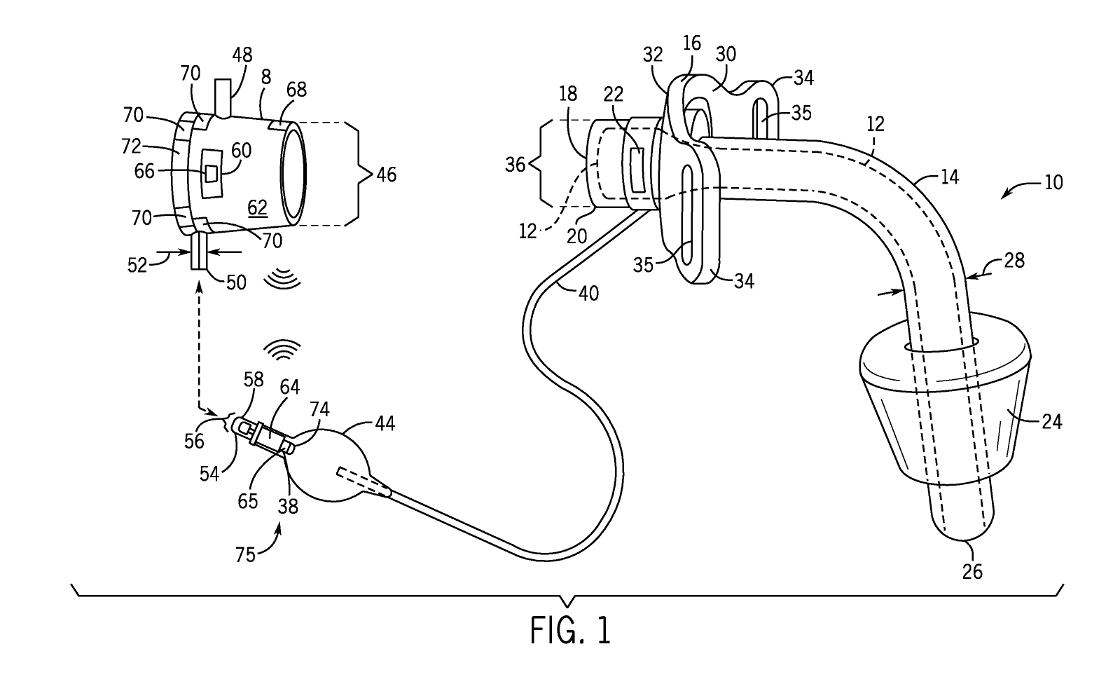

A tracheal tube according to a preferred embodiment is illustrated in FIG. 1. In the depicted embodiment, a speaking or phonation valve 8 is included in a tracheal tube system 10, as illustrated. The application of the speaking valve 8 to a tracheostomy tube is apt, however, insomuch as such tubes tend to be worn for longer periods of time, and thus the speaking valve 8 may be used to enable speech even when the tracheal tube is inserted in a patient. The tube system 10 may additionally include a removable and/or disposable inner cannula 12 shown disposed inside of an outer cannula 14, useful in maintaining a clean ventilation circuit.

The outer cannula 14 is illustrated extending both distally as well as proximally from a flange member 16. The inner cannula 12 may be introduced through an opening 18 of an end connector 20 and disposed inside of the outer cannula 14. During intubation, a tracheal tube assembly 22 including the inner and outer cannulae 12, 14 is placed through an opening formed in the neck and trachea of a patient, and extending into the patient airway. The tube assembly 22 embodiment illustrated in the figures includes a sealing cuff 24, although in practice a wide range of tube designs may be used, including tubes having no cuffs or tubes having multiple cuffs around the outer cannula 14.

In use, the sealing cuff 24 may be inflated so as to expand and contact the patient's airway. The inner cannula 12 in the illustrated embodiment may then form the sole conduit from which liquids or gases, including medications, may enter through the proximal opening 18 an exit through a distal opening 26. The cannula has an outer dimension 28 allowing it to fit easily through an incision made in the neck and trachea of the patient. In practice, a range of such tubes may be provided to accommodate the different contours and sizes of patients and patient airways. Such tube families may include tubes designed for neonatal and pediatric patients as well as for adults. By way of example only, outer dimension 28 of the tube 14 may range from 4 mm to 16 mm.

In one embodiment, the outer cannula 14 enters the flange member 16 along a lower face 30 and protrudes through an upper face 32 of the flange member 16. When in use, the face 30 will generally be positioned against the neck of a patient, with the cannula extending through an opening formed in the neck and trachea. A pair of side wings or flanges 34 extend laterally and serve to allow a strap or retaining member to hold the tube assembly in place on the patient. In the illustrated embodiment, apertures 37 are formed in each side flange 34 to allow the passage of such a retaining device. In many applications, the flange member 16 may be taped or sutured in place as well.

The end connector 20 is formed in accordance with industry standards to permit and facilitate connection to ventilating equipment (not shown) and to the speaking valve 8. By way of example, standard outer diameters may be provided as indicated at reference numeral 36 that allow a mating connector piece to be secured on the connector shown. By way of example, a presently contemplated standard outer diameter (OD) 36 accommodates a 15 mm connector, although other sizes and connector styles may be used. In use, then, air or other gas may be supplied through the connector and the inner cannula 12, and gases may be extracted from the patient. For example, the tube system 10 may be inserted into the patient's airways, and the cuff 24 may then be inflated through an inflation valve 40 fluidly coupled to an inflation lumen 42. A pilot balloon 44 may then indicate that air is in the cuff 24, thus sealing the patient's airway. Once the tracheal tube is positioned and secured, a ventilator may be coupled to the end connector 20.

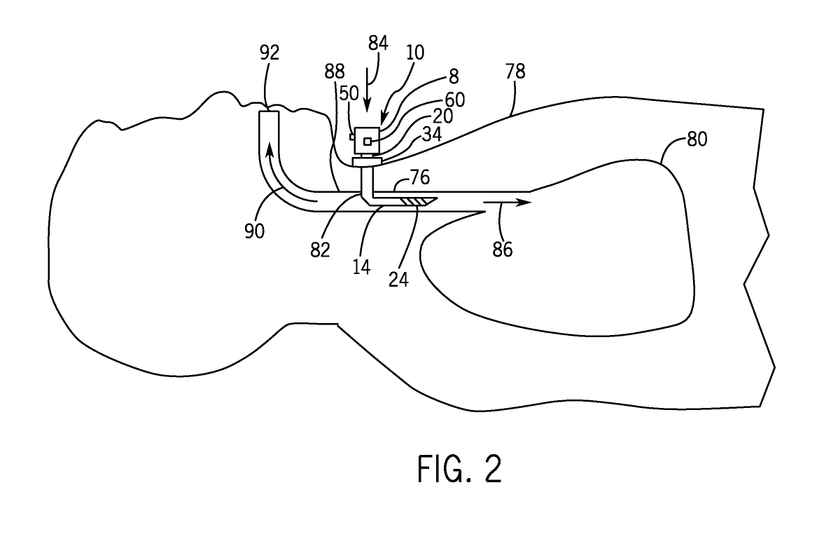

The speaking valve 8 may include an inner diameter (ID) 46 sized to mate with the end connector 20. In use, the speaking valve 8 is disposed over the end connector 20 and used to provide for one-way air intake into the patient's airway, as described in more detail below with respect to FIG. 2. In one embodiment as shown, the speaking valve 8 may include a supplemental oxygen port 48 useful for fluidly coupling the speaking valve 8 to a oxygen supply, such as an oxygen canister. Accordingly, supplemental oxygen may be delivered to the patient during use of the speaking valve 8. Also depicted is a mechanical cuff deflator 50 having an OD 52. In use, the mechanical cuff deflator 50 may be inserted inside of a bore 54 having an ID 56 slightly larger than the OD 52 and used to abut against a deflation head 58. By using the mechanical cuff deflator 50 to depress the deflation head 58 of the valve 38, the cuff 24 may be deflated.

Additionally, or alternatively, electronic or electrical means may be used to deflate the cuff 24. For example, a communications module 60 may be disposed on or inside of a body 62 of the phonation valve 8. The communications module 60 may be communicatively coupled, for example, through wireless techniques, to an electronic deflation system 64 included in the valve 38. In one example, the communications module 60 may include a switch or pushbutton 66 suitable for activating a deflation signal. Upon receipt of the signal, the electronic deflation system 64 may deflate the cuff 24, by using, for example, an actuator 65. For example, the actuator 65 may be a solenoid valve, a linear motion valve, a rotary valve, a piezoelectric valve, and so on, suitable for enabling the exit of gas from the cuff 24 into the ambient environment.

In another embodiment, the pushbutton 66 may not be included, and instead, proximity between the communications module 60 and the electronic deflation system 64 may be used to trigger the deflation of the cuff 24. For example, the communications module 60 may constantly be transmitting a low level deflation signal, and upon receipt of the low level deflation signal, the electronic deflation system 64 may deflate the cuff 24. The strength of the low level deflation signal may be derived so that when the phonation valve 8 is at a desired proximity to the inflation valve 38, the electronic deflation system 64 may automatically deflate the cuff 24.

In yet another example, a sensor 68 may sense the coupling of the phonation valve 8 to the end connector 20. The sensor 68 may be a magnetic sensor (e.g., Hall effect sensor), a photoelectric sensor, an ultrasonic sensor, a photocell, and/or a proximity sensor suitable for detecting the placement of the phonation valve 8 onto the end connector 20. Once the sensor 68 senses the coupling of the phonation valve 8 to the end connector 20, the communications module 60 may then transmit the deflation signal so that the electronic deflation system 64 may deflate the cuff 24. It is to be noted that the pushbutton 66 may be combined with the use of the sensor 68 and/or the always on low level deflation signal.

Additionally, one or more LEDs 70 may be disposed, for example, on the body 62 and/or a cap 72 of the speaking valve 8, and communicatively coupled to the communications module 60. The LEDs 70 may be provided in different colors, or with the ability to change colors. For example, the color red may be displayed when the cuff 24 is inflated, and the color green may be displayed when the cuff 24 is deflated. In the depicted embodiment, a pressure sensor 74 may sense the inflation pressure of the cuff 24, and the electronic deflation system 64 may transmit a signal representative of when the cuff 24 is at a deflation pressure suitable for phonation. The components 38, 54, 58, 64, 65, including sensor 74, may be included in a cuff deflation device 75. The communications module 60 may turn on or change the colors displayed by the LEDs 70 based on the signal transmitted by the electronic deflation system 64. For signals representative of a deflated cuff 24, the LEDs 70 may display the color red, and for signals representative of an inflated cuff 24, the LEDs 70 may display the color green. Of course, other colors may be used, including colors representative of a partially deflated cuff 24 (e.g., the color yellow).

It may be beneficial to describe the use of the speaking valve 8 during phonation, accordingly FIG. 2 shows the tracheostomy system 10 that has been inserted into a trachea 76 of a patient 78. The tracheostomy system 10 provides controlled access to the lungs 80 of the patient 78 via a tracheostomy site 82 on the anterior portion of the neck. As depicted, the system 10 provides a fluid pathway to the lungs 80. The flange 34 is disposed near the proximal end of the cannula 14 and rests on the anterior portion of the neck to provide stability to the system 10. At the proximal tip of the cannula 14, the connector 20 provides a connection point for attaching additional airway accessories to the system 10. Such an accessory may be the speaking valve 8, which enables the patient 78 to speak and breathe independently while the system 10 is disposed in the patient 78. As detailed below, the speaking valve 8 may include mechanical means, electronic means, or a combination thereof, suitable for deflating the cuff 24 when the patient desires to speak.

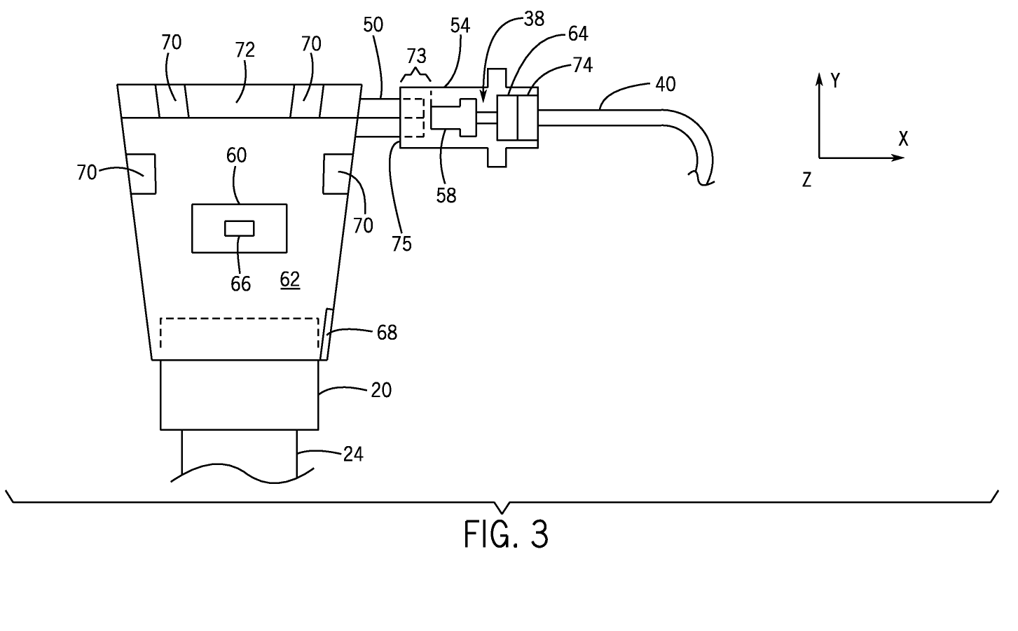

When the valve 8 is disposed onto the system 10, the mechanical cuff deflator 50 and/or the communications system 60 may be used to deflate the cuff 24, as mentioned above. To enable speaking, the valve 8 acts as a one-way check valve and allows only inhalation air, indicated by arrow 84, to travel through to the system 10 into the lungs 80. The inhalation air exits the distal end of the cannula 14 and enters the lungs 80, as indicated by arrow 86. When exhalation begins, the valve 8 may then block the air from exiting the patient 78 via the system 10, and because of the deflation of the cuff 24, forces the air around the system 10 to pass the larynx 88, as indicated by arrow 90. The larynx 88 houses vocal folds, which vibrate as the air (following arrow 90) flows past. Vibration of the vocal folds facilitates phonation. When speaking, the exhalation air exits the patient 78 via the mouth. Further details of the use of the mechanical cuff deflator 50 are described with respect to FIG. 3.

Turning now to FIG. 3, the figure is a side view depicting an embodiment of the phonation valve 8 coupled to the inflation valve 38 in the Y-Z plane. More specifically, the figure depicts the mechanical cuff deflator 50 disposed inside of the bore 54. When speech is desired, the valve 8 may be coupled to the end connector 20 to provide for one-way check valve functionality as described previously. Advantageously, the cuff 24 may be deflated, for example, by inserting the mechanical cuff deflator 50 inside of the bore 54 and pressing inwardly to abut the deflator 50 against the deflation head 58 of the valve 38. Depressing the deflation head 58 may then deflate the cuff 24. In one embodiment, the bore 54 may be manufactured out of a transparent or translucent material suitable for visualizing the contact and subsequent depression of the deflation head 58. The bore 54 may include a distance 73 between a proximal opening 75 of the bore 54 and the deflation head 58. The distance 73 may be smaller than the length of the mechanical cuff deflator 50, thus providing for the abutment of the mechanical cuff deflator 50 against the deflation head 58. Also depicted are the communications module 60 and the pushbutton 66 which may be used to electronically deflate the cuff 24, for example, by wirelessly transmitting deflation signals to the electronic deflation system 64.

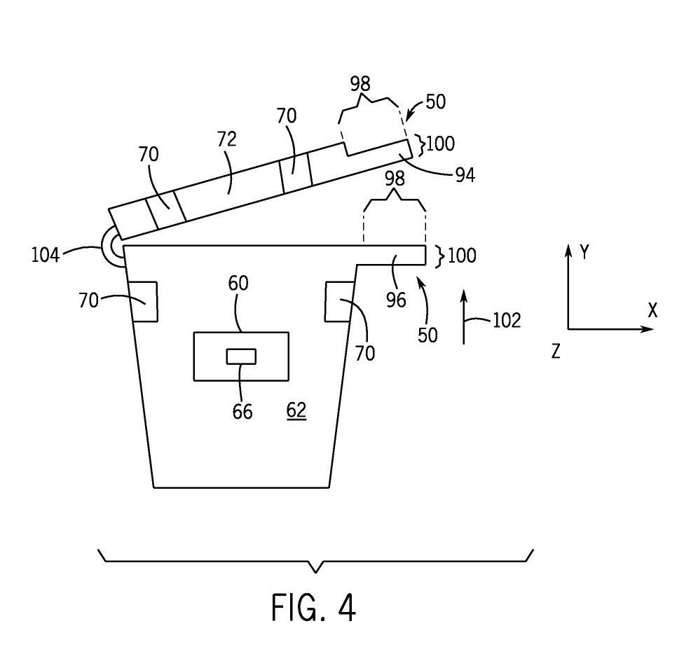

Also depicted is the sensor 68 which may be used to sense the coupling of the phonation valve 8 to the end connector 20, and of automatically triggering the transmission of deflation signals when the coupling is made. The sensor 74 is also depicted, useful in detecting pressure through the conduit 40. LEDs 70 are also depicted. The communications module 60 may use the pressures sensed by the sensor 74 to turn on/off or to change the colors of the LEDs 70 as a visual indication of cuff deflation. For example, the color green may be displayed by the LEDs 70 to denote a deflated cuff 24 or a cuff 24 that may be sufficiently deflated for phonation, while the color red may be displayed to denote an inflated cuff 24 or a cuff 24 that may be insufficiently deflated for phonation. Additionally, the mechanical cuff deflator 50 may include certain features useful in preventing the use of the phonation valve 8 if the cuff 24 is not properly deflate, as described in more detail below with respect to FIG. 4.

FIG. 4 is a side view taken on the X-Y plane of an embodiment of the valve 8 showing further details of the mechanical cuff deflator 50. In the depicted embodiment, the cuff deflator 50 is composed of an upper portion 94 and a lower portion 96, that, when contacting each other, form the cuff deflator 50. The upper portion 94 may be included as a section of the cap 72, while the lower portion 96 may be included as a section of the valve body 62. Each of the portions 94 and 96 may include a length 98 longer than the distance 73 (shown in FIG. 3) and suitable for abutting against the deflation head 58 when the portions 94, 96 are inserted into the bore 54. Likewise, the portions 94, 96 may include a width 100 (e.g., half the OD 52 shown in FIG. 1) smaller than ID 56 of the bore 54. It is to be noted that, in other embodiments, each portion 94, 96 may have different lengths and widths from the other portion.

The portions 94 and 96 may be separated, for example, by using a spring bias 102. The force 102 may be provided by, for example, a spring 104. In the depicted embodiment, the spring 104 is provided by molding or overmolding the spring 104 externally to the cap 72 and body 62 and used, for example, as the sole member or “hinge” connecting the cap 72 to the body 62. When the portions 94 and 96 are not disposed inside of the bore 54, the spring bias 102 is suitable for “opening” the cap 72 outwardly away from the body 62. Accordingly, the user or clinician may visually see that the portions 94, 96 have not been secured inside the bore 54. Accordingly, the portions 94 and 96 may be brought into contact with one another and then disposed inside of the bore 54, and then used to depress the deflation head 58, thus deflating the cuff 24. It is to be understood that the electronic components 60, 66, and 70 may additionally or alternatively be used to deflate the cuff 24. By providing for mechanical techniques additional to or alternative to the electronic techniques used for cuff 24 deflation, the embodiments described herein may enhance patient comfort, safety, and deflation efficiency.

In one example, the plurality of non-electronic components of valve 8 may be manufactured out of a material such as polyvinylchloride, a polyurethane, thermoplastic elastomers, a polycarbonate plastic, silicon, an acrylonitrile butadiene styrene (ABS), or a polyvinyl chloride (PVC), rubber, neoprene, or combination thereof. The electronic components (e.g., components 60, 66, 68, 70) may be housed inside cavities of the valve 8 and/or attached externally to the valve 8. Likewise, the non-electronic components of the tracheal tube assembly 22 may be manufactured of polyvinylchloride, polyurethane, thermoplastic elastomers, polycarbonate plastic, silicon, ABS, PVC, rubber, neoprene, or combination thereof, and the electronic components (e.g., 64, 74) may then be disposed on certain components of the tracheal tube assembly 22 (e.g., in or on the bore 54, inside cavities of the lumen 40, cannulae 12, 14, flange 34, attached to the lumen 40, cannulae 12, 14, flange 34).

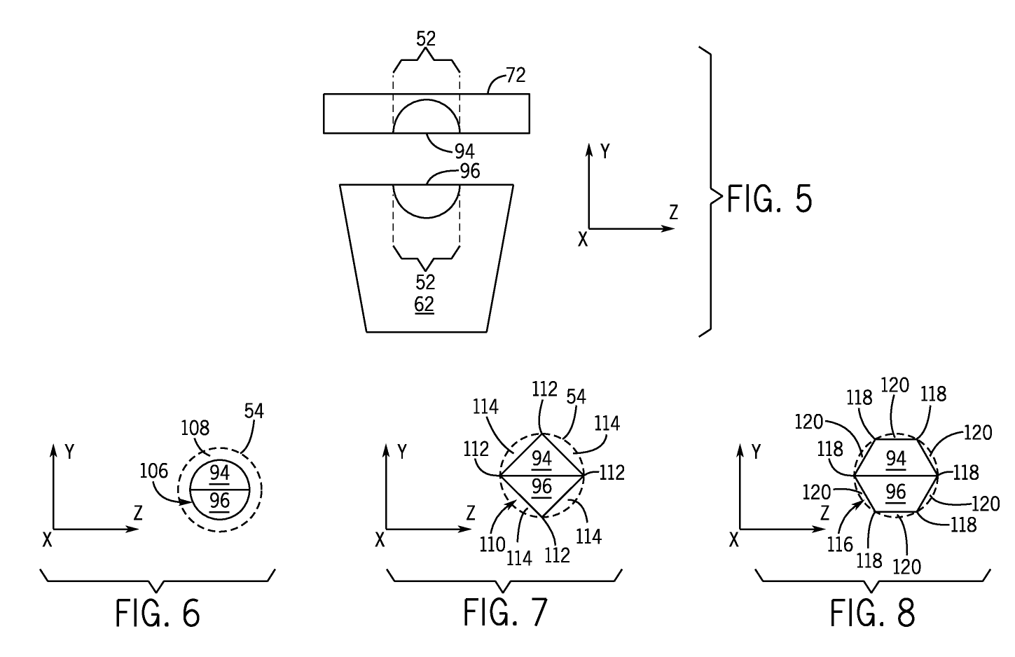

FIG. 5 is a frontal view taken on the Y-Z plane showing a semicircular shape for each of the portions 94, 96. The mechanical cuff deflator 50 may be formed in situ by contacting the portions 94, 96 against each other. When contacted together, the semicircular shapes may form a circle useful in more easily inserting the mechanical cuff deflator 50 inside of the bore 54. Because the circular shape may not include edges, the circular shape may be more effortlessly disposed into the bore 54, as depicted in FIG. 6. More specifically, FIG. 6 is a frontal view taken on the Y-Z plane depicting an embodiment of the portions 94 and 96, that when brought into contact with each other, form a circular shape 106 useful for insertion into the bore 54. As mentioned above, the circular shape 106 includes an OD smaller than the ID of the bore 54. Accordingly, an interstice or space 108 may be found between the circular shape 106 and the bore 54, which may enable the exit of inflation gas leaving the cuff 24 when the cuff 24 is deflated mechanically (e.g., by using the portions 94 and 96). However, the interstice 108 may be sufficiently small (e.g., having a surface area in the Y-Z plane of approximately 4 mm² or less) so that the portions 94 and 96 remain in place inside of the bore and do not easily slide outwardly away from the bore 54 yet gases may fluidly exit through the interstice 108.

The portions 94 and 96 may form other shapes when in contact with each other. For example, FIG. 7 depicts a parallelogram or square shape 110. The square shape 110 may advantageously enable the portions 94 and 96 to contact the bore 54 at points 112, thus more securely attaching the portions 94 and 96 to the bore 54. Interstices or spaces 114 may then provide for exit conduits through which the cuff 24 inflation gases may escape when the cuff 24 is mechanically deflated. The interstices 114 may each have a surface area in the Y-Z plane of approximately 4 mm² or less. Other shapes may additionally be provided, such as depicted in FIG. 8, suitable for enhancing the attachment of the portions 94 and 96 to the bore 54. For example, a hexagonal shape 116 may contact the bore's interior at points 118. Having a larger number of contact points, such as points 118, may increase the security of attachment of the portions 94 and 96 to the bore 54. The shape 116 may result in interstices or spaces 120 between the portions 94, 96 and the inside walls of the bore 54, suitable for the exit of cuff 24 inflation gases. Accordingly, the portions 94 and 96 may depress the deflation head 58, and gases may escape through the interstices 120. It is to be noted that, in other embodiments, the mechanical cuff deflator 50 may include only one of the portions 94 and 96, instead of the both portions. In this embodiment, the included portion may have any of the shapes 106, 110, 116, or a combination thereof. That is, the shapes 106, 110, 116, or a combination thereof, may be provided by a single one of the portions 94 or 96, when viewed on the Y-Z plane. Other shapes may be provided, including star shapes, oval shapes, multi-sided shapes (e.g., triangle, pentagon, heptagon, octagon), and so on.

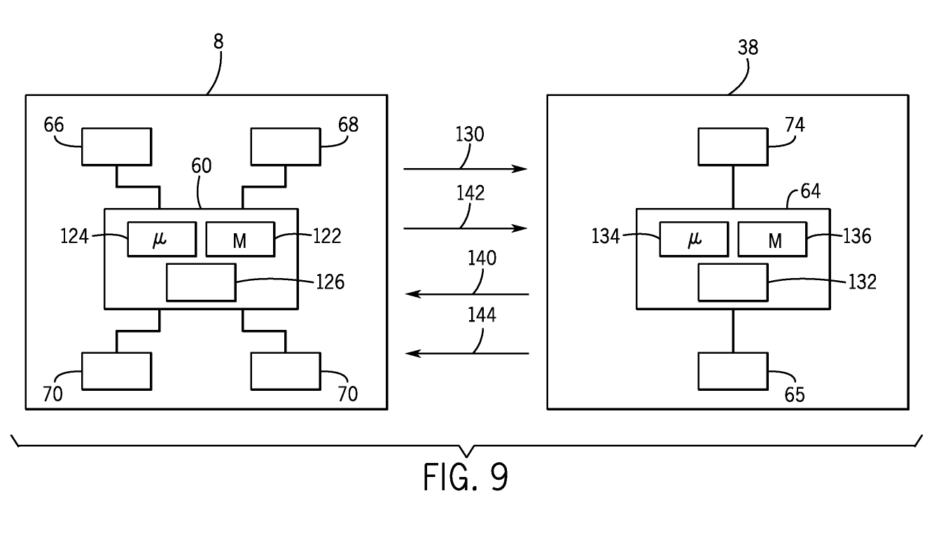

Turning now to FIG. 9, the figure is a block diagram depicting an embodiment of an electronic architecture showing the communications module 60 of the phonation valve 8 wirelessly coupled to the electronic deflation system 64 of the inflation valve 38. In the depicted embodiment, the communications module 60 includes a memory 122 useful in storing non-transitory computer readable instructions or code, and a microprocessor 124 suitable for executing the instructions stored in the memory 122. In other embodiments, the memory 122 and/or the microprocessor 124 may be replaced with custom circuitry suitable for communicating with the electronic deflation system 64, as described in more detail below. Further illustrated is a wireless module 126 suitable for sending and receiving wireless data. The wireless module 126 may include Bluetooth circuitry, Zigbee circuitry, WiFi (e.g., IEEE 802.11x) circuitry, near field communications (NFC) circuitry, and/or custom circuitry suitable for wireless transmitting and/or receiving signals. The communications module 60 may be provided as one or more integrated circuits (ICs) or other circuitry.

Also depicted are the pushbutton 66 and the sensor 68. In certain embodiments, either (or both) of the pushbutton 66 and the sensor 68 may be used to trigger a wireless signal 130. For example, the pushbutton 66 may be pressed when the phonation valve 8 is disposed or is about to be disposed onto the end connector 20, or the sensor 68 may detect that the phonation valve 8 has been disposed onto the end connector 20, thus triggering The wireless signal 130. The wireless signal 130 may then be received by a wireless module 132 included in the electronic deflation system 64, and processed by a processor 134. For example, the processor 134 may use non-transitory computer instructions or code stored in a memory 136 to process signals 130 received by the wireless module 132. The wireless module 132 may include Bluetooth circuitry, Zigbee circuitry, WiFi circuitry, NFC circuitry, and/or custom circuitry suitable for wireless transmitting and/or receiving signals. The electronic cuff deflation system 64 may be provided as one or more integrated circuits (ICs) or other circuitry.

Upon receipt of the signal 130, the electronic cuff deflation system 64 may deflate the cuff 24 by activating the actuator 65. For example, the actuator 65 may be a solenoid valve, a linear motion valve, a rotary valve, a piezoelectric valve, and so on, suitable for enabling the exit of gas from the cuff 24 into the ambient environment. For example, the actuator 65 may be disposed inside the bore 54 and used to transfer cuff 24 gases to the ambient environment. The pressure sensor 74 is also shown as communicatively coupled to the processor 134. Accordingly, the processor 134 may derive when the cuff is deflated (e.g., when pressure drops to ambient pressure). The processor 134 may additionally derive when the cuff 24 is inflated, or partially inflated/deflated. Based on these inflation derivations, the processor 134 may transmit a signal 140 representative of whether the cuff 24 is properly or improperly deflated for phonation. Upon receipt of the signal, for example, by using the wireless module 126, the processor 124 may turn on/off (or change colors) of the LEDs 70. As mentioned above, green may denote that the cuff 24 is ready for phonation (e.g., deflated for phonation), while red may denote that the cuff is not yet ready for phonation (e.g., fully inflated).

In some embodiments, signals 142 or the signals 144 may be “always on” signals. For example, the signal 142, like the signal 130, may also be transmitted to deflate the cuff 24. However, unlike the signal 130, the signal 142 may be transmitted all the time, battery power or other power source permitting. Likewise, the signal 144, like the signal 140, may be representative of the phonation status of the cuff 24, and transmitted all the time. In some embodiments, the signals 142 and 144 may be low strength signals, useful in conserving battery power. Low strength signals 142, 144 may additionally be used as proximity signals so that cuff deflation occurs when the phonation valve 8 and the cuff 24 and near each other (e.g., inside of approximately 2 feet). The signals 130, 132, 140, and 144 may be analog signals, digital signals, or a combination thereof. Additionally, authentication (e.g., “handshaking”) may be provided through the signals 130, 132, 140, and 144, for example, so that a specific valve 8 may only electronically deflate a specific tracheostomy tube assembly 22 having the cuff 24 that is “paired” to the valve 8, or a specific type of tracheostomy tube (e.g., pediatric, adult).

What is claimed is:

1. A tracheal tube system comprising:
a tracheal tube assembly comprising:
a cannula configured to be positioned in a patient airway;
a connector coupled to the proximal end of the cannula; and
a cuff disposed about the cannula; and
a speaking valve comprising means for deflating the cuff, wherein the speaking valve, the connector, and the cannula form a contiguous passageway for delivering air one-way into the patient airway when the speaking valve is disposed onto the end connector.

2. The system of claim 1, wherein the speaking valve comprises mechanical means for deflating the cuff, electronic means for deflating the cuff, or a combination thereof.

3. The system of claim 1, wherein the tracheal tube assembly comprises an inflation valve comprising a bore and a deflation head disposed inside of the bore and fluidly coupled to the cuff, wherein the deflation head is configured to deflate the cuff when depressed, and wherein the mechanical means for deflating the cuff comprises a mechanical cuff deflator configured to depress the deflation head.

4. The system of claim 3, wherein the mechanical cuff deflator comprises a first protrusion having a first protrusion width smaller than an internal diameter (ID) of the bore.

5. The system of claim 4, wherein the mechanical cuff deflator comprises a second protrusion having a second protrusion width, and wherein the first and the second protrusions form a shape having a shape width smaller than the ID when the first and second protrusions are brought into contact with each other.

6. The system of claim 5, comprising a spring having a bias configured to force the first protrusion away from the second protrusion.

7. The system of claim 6, wherein the speaking valve comprises a cap and a body, wherein the first protrusion is disposed on the cap and the second protrusion is disposed on the body, and wherein the spring connects the cap to the body.

8. The system of claim 5, wherein the shape comprises a circle, a square, a hexagon, or a combination thereof.

9. The system of claim 1, wherein the speaking valve comprises a communications module comprising means for transmitting a deflation signal, and wherein the tracheal tube assembly comprises an electronic cuff deflation system comprising means for deflating the cuff upon receipt of the deflation signal.

10. The system of claim 9, wherein the speaking valve comprises a light communicatively coupled to the communications module and wherein the electronic cuff deflation system comprises means to transmit a cuff status signal based on a pressure of the cuff, and wherein the communications module comprises means to receive the cuff status signal and to turn on or off the light based on the cuff status signal.

11. The system of claim 9, wherein the speaking valve comprises a sensor communicatively coupled to the communications module and configured to sense the disposition of the speaking valve onto the end connector, and wherein the communications module comprises means for transmitting the deflation signal based on the sensor.

12. The system of claim 9, wherein the speaking valve comprises a switch communicatively coupled to communications module and wherein the communications module is configured to transmit the deflation signal when the switch is activated.

13. A tracheal tube system comprising:
a speaking valve comprising:
a first protrusion comprising a first width and a first length, wherein the first width is smaller than a bore width of a bore included in a cuff deflation device, and the first length is longer than a distance between a proximal opening in the bore an a deflation head disposed in the bore, and wherein the speaking valve provides for one-way airway gas flow when in use.

14. The system of claim 13, comprising a body having the first protrusion, a cap, and a second protrusion disposed on the cap, wherein the second protrusion comprises a second width and a second length, wherein the second width is smaller than the bore width, and the second length is longer than the distance.

15. The system of claim 14, comprising a hinge coupling the cap to the body.

16. The system of claim 15, wherein the hinge comprises a spring bias configured to force the first protrusion away from the second protrusion.

17. A tracheal tube system comprising:
a speaking valve comprising:
a communications module comprising a wireless circuitry configured to transmit a wireless cuff deflation signal to deflate a cuff, wherein the speaking valve provides for one-way airway gas flow when disposed onto a tracheal tube assembly having the cuff

18. The system of claim 17, comprising the tracheal tube assembly having an electronic cuff deflation system, and wherein the electronic cuff deflation system is configured to deflate the cuff upon receipt of the wireless cuff deflation signal.

19. The system of claim 17, comprising the tracheal tube assembly having an end connector, wherein the speaking valve comprises a sensor communicatively coupled to the communications module and configured to sense a disposition of the speaking valve onto the end connector, and wherein the communications module is configured to transmit the wireless cuff deflation signal based on the sensor to deflate the cuff

20. The system of claim 17, comprising the tracheal tube assembly having an electronic cuff deflation system, wherein the speaking valve comprises a light communicatively coupled to the communications module and wherein the electronic cuff deflation system is configured to transmit a cuff status signal based on a pressure of the cuff, and wherein the communications module is configured to receive the cuff status signal and to turn on or off the light based on the cuff status signal.
